(19) United States
(12) Patent Application Publication
(10) Pub. No.: US 2023/0153862 A1
(43) Pub. Date: May 18, 2023

(54) REAL-TIME INTERACTIVE ADVERTISEMENTS PLACED ON A MOVING VEHICLE

(71) Applicant: William Frederick Vartorella, Camden, SC (US)

(72) Inventor: William Frederick Vartorella, Camden, SC (US)

(21) Appl. No.: 17/897,558

(22) Filed: Aug. 29, 2022

Related U.S. Application Data
(63) Continuation-in-part of application No. 17/524,802, filed on Nov. 12, 2021, now abandoned.

(51) Int. Cl. G06Q 30/02; G09F 21/04

(52) U.S. Cl. CPC G06Q 30/0266; G06Q 30/0257; G09F 21/048

(57) ABSTRACT

A system for placing a real-time interactive advertisement on, inside, or in the general vicinity of a moving vehicle, during a live feed, wherein a user has the capability to pause and then click on the provided HTML hyperlinks with the goal of a user learning more about an advertisement.

This application is a continuation-in-part of U.S. non-provisional patent application Ser. No. 17/524,802 filed on Nov. 12, 2021, which is incorporated herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a system for placing a real-time, interactive advertisement on, inside, or in the general vicinity of a moving vehicle via a digital image or material.

### Description of Related Art

The placement of advertising on moving vehicles has become an extremely beneficial marketing tool for companies to advertise and market their product(s) outside and inside of a moving vehicle. In recent years, this has been done by use of a digital imprinted vinyl wrap and is usually a continuous single wrap on the outside of the vehicle. A problem with advertisements located on the outside or inside a vehicle is that in a real-time situation, the vehicle is moving and advertisements, logos, etc., may be hard to view, especially at high speeds. Since people might not have something to write the advertisement down on, the advertisement effectiveness is greatly diminished. In addition, since the vehicles may be moving extremely fast, it is frequently difficult to see or read the advertisements, much less comprehend what you are looking at.

There have been attempts to address advertising through video playback, but none of these attempts address or are capable of interactive digital advertising with real-time moving billboards (the moving vehicle).

### BRIEF SUMMARY OF THE INVENTION

The present invention relates to a system for advertising on a moving vehicle where interactive advertisements are digitally placed on, inside, or in the general vicinity of the vehicle. The live-action advertisement can be paused and the advertising is digitally interactive, links to a web site, hyperlink, or other online information, thus allowing a user to interact with the advertisement before it disappears.

Accordingly, in one embodiment, there is a system for real-time interactive digital advertising which appear to be on, inside, or in the general vicinity of a moving vehicle comprising:
a) a real-time moving vehicle, the moving vehicle where in there is a live feed of the vehicle, inside the vehicle, and in the general vicinity of the moving vehicle;
b) a user computer capable of allowing a user of the system to watch the live feed of the moving vehicle and pause live action;
c) a device capable of real-time, stop-action pause of the live feed on the user computer;
d) a digital image or material which appear to be on, inside, or in the general vicinity of the moving vehicle having a pixilated clickable image which includes HTML language which can be read by the user computer; and
e) wherein the HTML acts as a hyperlink to another website, game, advertisement, camera angle, or interview, when clicked on by user.

### BRIEF DESCRIPTION OF THE DRAWINGS

FIG. 1a is a side view perspective of the present invention showing a moving vehicle on a computer screen with digital interactive advertisements.

FIG. 1b is a perspective view of the present invention showing the advertisement on a computer after the HTML hyperlink has been activated.

FIG. 2 is a perspective view of the cockpit of a moving vehicle showing an advertisement placement of the present invention on the dash of the moving vehicle.

FIG. 3 is a flow chart of the method of use of the present invention.

FIG. 4 is a flow chart of the method of using a Facial Recognition Software system.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “real-time advertising” refers to advertising during a live feed of a moving vehicle. The term advertising is used in a broad sense so as to not only include product placement advertising, but also any other event, place, organization, logo, website, game, camera angle, interviews, and the like, to be reached through the digital interactive advertising and then provide a hyperlink for information relating to the advertisement.

As used herein, the term “on, inside, or in the general vicinity” refers to placements of advertising on the outside of the vehicle, inside of the vehicle, or in the general vicinity of the moving vehicle. The advertisements are placed digitally or on a material, such as a wrap, sticker, or decal, via a digital printer with embedded HTML, which may also be applied to a painted surface. When the vehicle is moving, and when the live action is paused/stopped, the digital advertisement remains as an active HTML hyperlink. In one embodiment, the advertisement is placed on a HTML-configured vinyl wrap with pixels that are clickable. The pixels with HTML are placed on the wrap (via printing) to become clickable. In another embodiment, the material is a sticker, decal, and the like.

As used herein, the term “in the general vicinity of the moving vehicle” refers to the area near or surrounding the vehicle. For example, the stands, crowds, billboards, event personnel, concession stands, entertainment props, such as, ramps, and the like, and in the vicinity. The term also refers to the surrounding area around the moving vehicle that is associated with the vehicle because the vicinity is on the same live feed as the vehicle during a race. For example, in a car race, the pit, the tracks, race track field, race team personnel, equipment for moving vehicles, such as, tires, balloons, tools, and the like, are in the vicinity of the moving vehicle.

As used herein, the term “moving vehicle” refers to any powered vehicle moving during a live feed. In one embodiment, the vehicle is a race vehicle, which would be in a race (e.g., race car, race boat, racing motorcycle, racing plane, racing balloons, and the like). In another embodiment, the vehicle is a non-racing vehicle that moves from one place to the next, such as a balloon, snowboard, aircraft, car, golf cart, and the like.

As used herein, the term “real-time moving vehicle” refers to a live feed relating to a moving vehicle that is broadcast and transmits to a computer for viewing, allowing the user to click on any number of the HTML hyperlinks associated with the advertisement.

As used herein, the term “live feed” refers to the seeing and hearing of the moving vehicle live, which is occurring at the same time as it is being viewed by a user, including streaming, Cable TV, emerging technologies, and the like.

As used herein, the term “computer” refers to, for example, one or more servers, motherboards, processing nodes, laptops, tablets, personal computers (portable or non-portable), personal portable information terminals, smart phones, smart watches, Smartband, cell phone or mobile phone, another mobile device having at least a processor and memory, a video game system, an augmented reality system, a holographic projection system, a television, a wearable computer system, an Internet of Things node, and/or it may also include other device(s) providing one or more processors that are at least partially controlled by instructions. The instructions may be in the form of firmware or other software in memory and/or special circuitry.

As used herein, the term “real-time stop-action pause” refers to the ability to pause a live broadcast of a moving vehicle being viewed on a computer. This is required to preserve the HTML hyperlink associated with the real-time digital interactive advertisements and make it easier and more reasonable to click the HTML hyperlink.

As used herein, the term “real-time digital interactive advertisements” refers to an advertisement, a logo, symbol, figure, word(s), and the like, which appear to be on, inside, or in the vicinity of the moving vehicle The real-time digital interactive advertisements are in the form of pixels as a pass-through, via click to a database. In one embodiment, the use of artificial intelligence (Facial Recognition Software) is used to support the identification of advertising sponsor logotypes. They can advertise anything such as a product, a service, general information, race information, and the like. The real-time digital interactive advertisement, for example, can be produced by blue screen technology, HTML hyperlink, pixel images, wireless technology, artificial intelligence (Facial Recognition Software), or the like, but must also preserve a HTML hyperlink associated with the digital interactive advertisement when the live feed is paused. The real-time digital advertisement serves as a clickable image that also acts as a HTML hyperlink.

A clickable image is an image that also acts as an HTML hyperlink. In digital imaging, a “pixel” or picture element is the smallest controllable element of a picture represented on a smart screen in a raster image. In color imaging systems, a color is typically represented by three or four component intensities (red, green, blue or cyan, magenta, yellow, and black). This is important, as these pixel images, as illustrated in the drawings, may include HTML language which can be read-interpreted by a computer device on a screen (Smart TV, cellular phone, etc.). Clicking on any part of the image (here, meaning a pixel embedded logotype, or the like) will redirect the user to another the advertisement. In one embodiment, because of the HTML-configured vinyl wrap described below, sponsor logos or a portion of a sponsor logo, may be clicked and the viewer is led to a database with enriched content of interest to the viewer.

Simple HTML, placed on a HTML-configured vinyl wrap is used to create an image as clickable on a digital device, just as a text link. This technology allows the moving vehicle to then become a moving website. The HTML tags used to display images are called img tags. These have an attribute called System Resource Controller (SRC) that points to the URI (Universal Resource Identifier) or the path to the image file. The HTML tag that is created that creates a link is called a hyperlink tag or just an <a> tag. The <a> tag takes a mandatory argument called Hypertext Reference (HREF) which points to the target link or target URI. The text between the start and the end of <a> tag is the clickable part of the link. In order to make an image clickable, all that has to be done is to use both of these tags together. In order to make the sponsor image clickable, the <img> tag will be surrounded with the <a> tag. Essentially, it replaces the text, which is the clickable part of the link, with the sponsor image itself. With the above code, the resulting image is now a link and clicking on it will load the page referred to by the HREF attribute which is/path/to/target.html, in this example. In the present invention, the code is embedded in the digitized image, which is transferred to the vinyl wrap/livery of the race car.

An increasing number of race cars are wrapped in a special sheet of 3M™ vinyl wrap, nose-to-tail, sponsor(s)-specific. Vendors use computers that can easily modify the design and use digital ink jet printers to input the logotype on vinyl in 90 minutes. NASCAR® has actually moved individual race car numbers forward, off what is traditionally a door on your daily-driver, to provide more livery space on the “wrap” for additional advertising. These wraps, known as the “wave of the future” for racing series, are already employed by some 50% of NASCAR® racers-on-the-grid. As the wraps weigh on average about nine pounds less than a painted livery with full decals, they also provide a weight and potential aerodynamic advantage to teams. Important to the present invention is that the pixels can be exploited as described above and become interactive on the computer screen through embedded HTML, easily done when the sponsor logotypes are digitally scanned for the race car “wrap”. Using artificial intelligence (AI) through Facial Recognition Software, even traditional pixels displayed in the cockpit of the race car (or billboards or images in pit lane, or the like) can be interpreted and linked to a recognition database and HTML hyperlink to websites for additional viewer content. Exploring how Facial Recognition Software works and how it's used to identify sponsor logotypes as faces is an important embodiment of the present invention. In the present invention: 1. Image is captured. 2. Using the special pixels of the logotype described herein (instead of search for human eye), the “captured logotype” is, 3. Converted to a grayscale and cropped into a predetermined size (here, the size of the sponsor logotype). 4. The logotype image is converted to a sponsor “template” (created by a sanctioning body, a team, or a racetrack, or the like, for example) and compared to other sponsor logotypes on file. 5. Once matched, the algorithm accesses the specific logotype's database and the viewer chooses specific content via click.

This shell application of a hybrid form of artificial intelligence (Facial Recognition Software) to a paused image of a vehicle, allows partially visible sponsor logotypes (e.g., near the wheel well, in the cockpit, or on an oblique billboard) to be identified through comparison with the database, and once the identification is made, will enable the viewer to engage (click) with enriched content. The system of the present invention provides exposure for small, often hidden sponsor logos by identifying them as the cursor moves over the vehicle and enabling click-through in the application. The ability of the AI to read the only partially-visible logotypes, often indiscernible by the human eye, under the speed of a moving vehicle or partially-obscured billboard (also to be added to the database) is a major, disruptive advancement and serves to meet the “substantial” test through the multiplier effect of revenue stream. Again, in-kind, suppliers notoriously have their logos applied in obscure, often inopportune places. Now, for the first time we have value-added to the mere “donation” of some widget by a sponsor (brake line, etc.) with inclusion and attachment within a database. This is a huge advancement in race car advertising technology.

As used herein, the term “digital overlays” refers to a live event broadcast, wherein an image, information text, or the like, is added as an additional layer to the live broadcast a user is viewing on the computer.

As used herein, the term “other objects” refers to other stationary or moving objects that may be in view or in the vicinity during the live feed, such as a parked race car, advertisements in the stands, the pit, crowds, and the like.

As used herein, the term “hyperlink” refers to the ability to click on the live feed or paused interactive advertisement and be relocated to a new area of content or another website, live location, selection of items, and the like.

In short, the present invention works in the following embodiment: 1. Teams, sanctioning bodies, race tracks, or the like, scan and digitize sponsor logotypes as pixels. 2. Using the described vinyl wrap and the digitized logotypes via a digital printer, the wrap-shop specialists create and apply the digital livery to the racing vehicle. 3. Sanctioning bodies, et al. offer users on user computers a form of “freeware” that includes a version of Facial Recognition Software that can read, compare sponsor logotypes or partial views with a database that becomes viewer interactive with a simple click (several companies offer “apps” of Facial Recognition Software easily adapted for the present invention's use). 4. User viewing a race in digital format sees a logotype on a racing vehicle, pit lane, racing venue, driver helmet, cockpit, or the like, and clicks the standard pause-and-play button. 5. The viewer rolls the cursor (or other pointing/selection device) over the partial or complete logotype and clicks. 6. Facial Recognition Software matches the logotype with the database and connects the viewer to a product advertisement, an event, place, organization, logo, website, game, different camera angles, interviews, or the like. 7. Upon completion of viewing/downloading special content/promotions, or the like, the viewer re-clicks the standard pause-and-play button and is rejoined to the race where it left off.

The result is: a moving billboard (e.g., a race car) becomes a moving website/database-in, and of itself, a multiplier, and a disruptive use of the known uses of “parts”, combined with Facial Recognition Software alterable and exploitable for totally-unrelated and yet unpredicted use.

### DRAWINGS

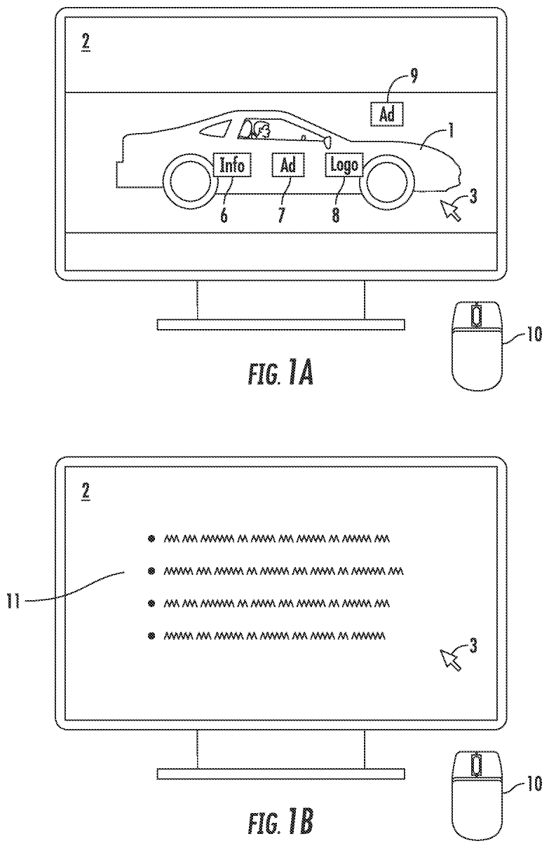

Now referring to the drawings, FIG. 1a is a side perspective view of the present invention. In this view, a moving vehicle, in this embodiment, is a live video of a race car 1 shown on computer screen 2. Also, on computer screen 2 is computer arrow/pointer/cursor 3 for positioning and clicking on real-time digital advertisements 6, 7, 8, and 9 with computer mouse 10 to activate pixels with a hyperlink associated with the real-time digital interactive advertisements 6, 7, 8, and 9. In one embodiment, the live stream is paused in order to make it easier to click on the real-time digital interactive advertisements 6, 7, 8, and 9.

FIG. 1b shows the computer screen 2 after the hyperlink has been activated and subsequently, has taken the user to a page 11 where the user encounters a list of items.

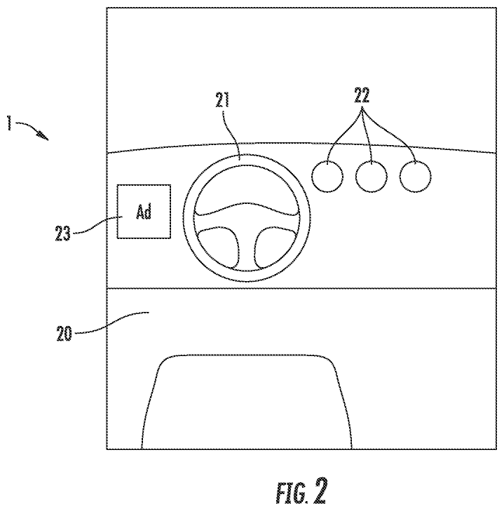

FIG. 2 shows the cockpit 20 of race car 1. A steering wheel 21 and gauges 22 are shown. A real-time digital interactive advertisement 23 is shown on the live feed. Again, the hyperlink is activated by clicking on the real-time digital interactive advertisement 23 where the live video can be paused to make viewing advertisements easier.

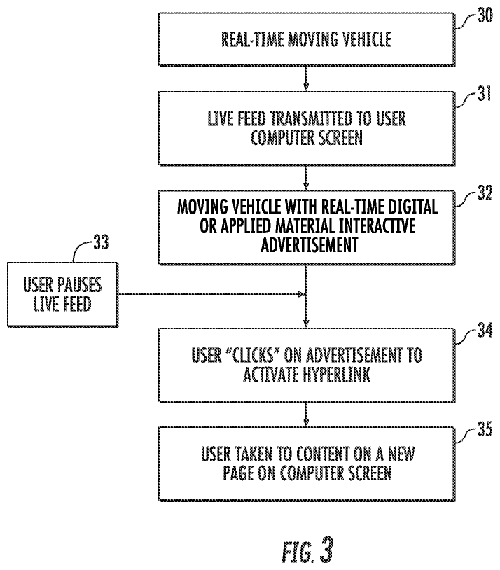

FIG. 3 is a flow chart of the method of using the real-time digital interactive advertisement system. The system starts with a real-time moving vehicle 30, wherein a live feed is transmitted to the user computer screen 31. The inside, outside, and general vicinity of the moving vehicle are fitted with real-time digital interactive advertisements 32. The user is then able to pause the live feed 33 and click on the advertisement to activate the hyperlink 34. Lastly, the user is relocated to a new area of content or a new page 35.

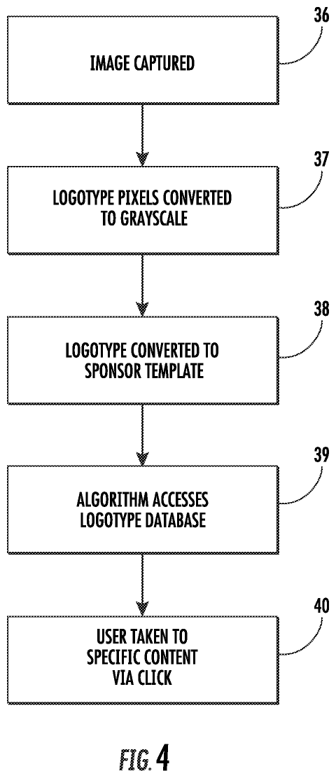

FIG. 4 is a flow chart of the method of using a Facial Recognition Software system to identify sponsor logotypes in one embodiment of the present invention. An image is captured 36. Using special pixels of logotypes, the captured logotype image is converted to grayscale 37 and cropped into a predetermined size. The logotype image is then converted to a sponsor template 38 and, when clicked on by the user, the logotype image is compared to other sponsor logotypes (for purpose of identification) that are within a file/database of advertisements, events, places, organizations, logos, websites, games, camera angles, interviews, and the like. Once the logotype is matched to an advertisement, or the like, the algorithm accesses the specific logotype's file/database 39 and the viewer chooses specific content based on the specific logotype's content. When the user chooses a specific advertisement, or the like, to view, the user will click on the content and is taken to the specific content 40 relating to the advertisement, or the like.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A system for a real-time interactive digital image which real-time interactive digital image appears to be on the inside, on the outside, or in the general vicinity of a moving vehicle by a user comprising:
a) a real-time moving vehicle, the moving vehicle wherein there is a live feed of at least one of the outside of the moving vehicle, the inside of the moving vehicle, and in the general vicinity of the moving vehicle;
b) a user computer capable of allowing the user to watch the live feed and pause live action;
c) a device capable of real-time, stop-action pause of the live feed on the user computer;
d) the digital image which appears on the user computer live feed to be on at least one of on the outside of the moving vehicle, on the inside of the moving vehicle and, in the general vicinity of the moving vehicle, the digital image which includes HTML having machine readable pixels which can be read by the user computer facial recognition software when clicked on; and
e) wherein the HTML machine readable pixels acts as a hyperlink to another website, game, advertisement, camera angle, or interview, when the computer clickable image is clicked on by the user of the computer live feed.

2. (canceled)

3. (canceled)

4. (canceled)

5. The system for a real-time interactive digital image according to claim 1, wherein the moving vehicle is a race car.

6. The system for a real-time interactive digital image according to claim 1, wherein the HTML machine readable pixels are on a vinyl wrap.
